(19) United States
(12) Patent Application Publication
(10) Pub. No.: US 2001/0022654 A1
(43) Pub. Date: Sep. 20, 2001

(54) Method & apparatus for determining red blood cell deformability by imaging red blood cells while applying vibratory energy

(75) Inventors: Kenneth Kensey, Malvern, PA (US); William N. Hogenauer, Gilbertsville, PA (US)

Correspondence Address: CAESAR, RIVISE, BERNSTEIN,, COHEN & POKOTILOW, LTD., 12TH FLOOR, SEVEN PENN CENTER, 1635 MARKET STREET, PHILADELPHIA, PA, 19103-2212, US

(73) Assignee: Visco Technologies, Inc., Exton, PA (US)

(21) Appl. No.: 09/836,103

(22) Filed: Apr. 17, 2001

Related U.S. Application Data
(63) Continuation-in-part of application No. 09/631,046, filed on Aug. 1, 2000.
Application No. 09/631,046 is a continuation of application No. 09/383,177, filed on Aug. 25, 1999, now Pat. No. 6,261,244.
Application No. 09/383,177 is a continuation of application No. 08/919,906, filed on Aug. 28, 1997, now Pat. No. 6,019,735.

(51) Int. Cl. G01N 33/49

(52) U.S. Cl. 356/39

(57) ABSTRACT

A method and apparatus for determining the deformability of red blood cells by illuminating a sample of red blood cells that are subjected to vibration and then using image recognition analysis to compare these vibrated red blood cells to red blood cells that are not experiencing vibration. The amplitude and frequency of vibration at which deformation or distortion occurs can also be determined.

### RELATED APPLICATIONS

This application is a Continuation-in-Part of Co-Pending application Ser. No. 09/631,046, filed Aug. 1, 2000, which in turn is a Continuation of application Ser. No. 09/383,177, filed Aug. 25,1999, which in turn is a Continuation of application Ser. No. 08/919,906, filed Aug. 28, 1997 (now U.S. Pat. No. 6,019,735, issued on Feb. 1, 2000), and all of which are entitled VISCOSITY MEASURING APPARATUS AND METHOD OF USE, and all of which are assigned to the same Assignee as the present invention, namely Visco Technologies, Inc. and all of whose entire disclosures are incorporated by reference herein.

### FIELD OF THE INVENTION

The invention pertains to methods and apparatus for determining the deformability of red blood cells, and more particularly, to methods and apparatus for determining the deformability of red blood cells using image analysis and the application of vibratory energy.

### BACKGROUND OF INVENTION

In U.S. Pat. No. 6,019,735 (Kensey et al.), there is disclosed a device for reducing the blood viscosity of a living being by applying vibratory energy to the living being. Furthermore, U.S. Pat. No. 6,019,735 (Kensey et al.) also discloses a device for determining the deformability of RBCs by using a plurality of lumens having different diameters that are less than the average diameter of a typical RBC and which is placed in the circulating blood of the living being; it is then seen which RBCs flow through which particular lumen.

However, there remains a need to determine the deformability of RBCs by applying vibratory energy to RBCs, using an image analysis of RBCs subjected to such vibratory energy and then comparing the distortion of the RBCs against a RBC that is not subjected to vibration.

### SUMMARY OF THE INVENTION

A method for determining the deformability of red blood cells (RBCs) of a living being, said method comprising the steps of: (a) providing a sample of RBCs from the living being; (b) applying vibratory energy to the RBCs, (c) monitoring any distortion in the RBCs; (d) monitoring the amplitude and frequency of the applied vibratory energy; and (e) comparing any distortion of the RBCs against an RBC that is not subjected to the vibratory energy.

An apparatus for detecting the deformability of red blood cells (RBC). The apparatus comprises: a specimen support for supporting a sample of RBCs thereon; a source of vibration coupled to the specimen support for applying vibratory energy to the specimen support; an illumination source for illuminating the sample of RBCs on the specimen support; an imaging means for obtaining an image of at least one RBC in the sample of RBCs; a computer, coupled to the imaging means and to the vibration source and wherein the computer has a memory comprising: an image of an RBC that has not been subjected to vibration; and image recognition software that compares the image of an RBC that has not been subjected to vibration with the image of at least one RBC in the sample of RBCs to determine the distortion of the at least one RBC caused by vibration.

### DESCRIPTION OF THE DRAWINGS

FIG. 1 is a block diagram of the present invention; and

FIG. 2 is a depiction of how a distorted RBC is compared against an undistorted RBC using image recognition software or machine vision software.

### DETAILED DESCRIPTION OF THE PREFERRED EMBODIMENTS

Referring now in detail to the various figures of the drawing wherein like reference characters refer to like parts, there is shown at 20 the system for determining the deformability of RBCs using vibratory energy and image analysis, hereinafter “the system 20.” The system 20 basically comprises an illumination source 22, a specimen support 24, a vibration source 26, a high resolution camera lens 28 and a computer 30 including image recognition software or machine vision software.

The illumination source 22 is positioned underneath the specimen support 24 for illuminating the specimen of RBCs 31 which is trapped between a pair of glass or otherwise transparent, slides 32A/32B. The slides 32A/32B, including the specimen of RBCs, are positioned in the specimen support 24 for analysis. The vibration source 26 is coupled to the specimen support 24 in order to vibrate the specimen support 24 with vibratory energy having an amplitude and frequency. The vibration source 26 includes an electrical signal output 34 (e.g., RS-232 compatible, etc.) that is conveyed to the computer 30. Thus, the computer 30 receives the instantaneous vibration amplitude and frequency information corresponding to the vibratory energy being applied to the specimen support 24. As an example, if the vibration source 26 sweeps a range of vibration frequencies during the analysis, the computer 30 always knows what amplitude/frequency of vibration are being applied as the image recognition software, or machine vision software, is conducting its image analysis.

The high resolution lens 28 obtains a high resolution image of the illuminated specimen 31 and provides that image to the computer 30 for the analysis. The high resolution lens 28 may comprise a charge-coupled device (CCD) or may comprise a CMOS (complimentary metal oxide semiconductor) image sensor. It should be understood that the it is within the broadest scope of this invention to include both analog and digital lens/camera technology and is not limited to either one. In any case, the lens 28 provides an electronic version of the high resolution image to the computer 30.

The computer 30 uses the image recognition software or machine vision software to compare the image being transmitted from the high resolution lens 28 to a pre-stored image of an RBC that is not subjected to any vibration. FIG. 2 depicts this comparison concept. The upper item 36 is an isometric view of an RBC that is not subjected to vibration and which has been pre-stored in the memory of the computer 30. The lower item 38 is an isometric view of the current, high resolution image of an RBC in the specimen 31 that is being subjected to the vibration. As can be seen in FIG. 2, the outer perimeter 40 of the RBC is very distorted as compared to the outer perimeter 42 of the RBC 36. The image recognition software or machine vision software does a point-to-point analysis along these perimeters 40 and 42 to determine the amount of distortion occurring. Furthermore, since the computer 30 also knows the current amplitude and frequency levels of the vibration, the computer 30 can also determine the particular amplitude/frequency of vibration where RBC distortion begins.

Although not limited to the following image recognition software, or machine vision software applications, the following patents/patent application provide exemplary systems that can be used with the computer 30 and their entire disclosures are incorporated by reference herein:

U.S. Pat. No. 4,471,043 (Bacus)

U.S. Pat. No. 5,031,228 (Lu)

U.S. Pat. No. 5,235,522 (Bacus)

WO 93/16436 (Nelson et al.)

Furthermore, these exemplary systems can implement the cell imaging techniques disclosed in the following references to determine the amount of distortion:

“Energy-Filtering Transmission Electron Microscopy of Biological Specimens” by Bruijn et al.

“Determination of Pore Structure by Stereological Measurements” by Fischmeister

“A Software Laboratory for Visual Inspection and Recognition” by Gini et al.

“Comparative Test of Methods to Determine Particle Size and Particle Size Distribution in the Submicron Range” by Lange

“Shape Discrimination Using Fourier Descriptors” by Persoon

“The Analysis of Cell Images” by Prewitt et al.

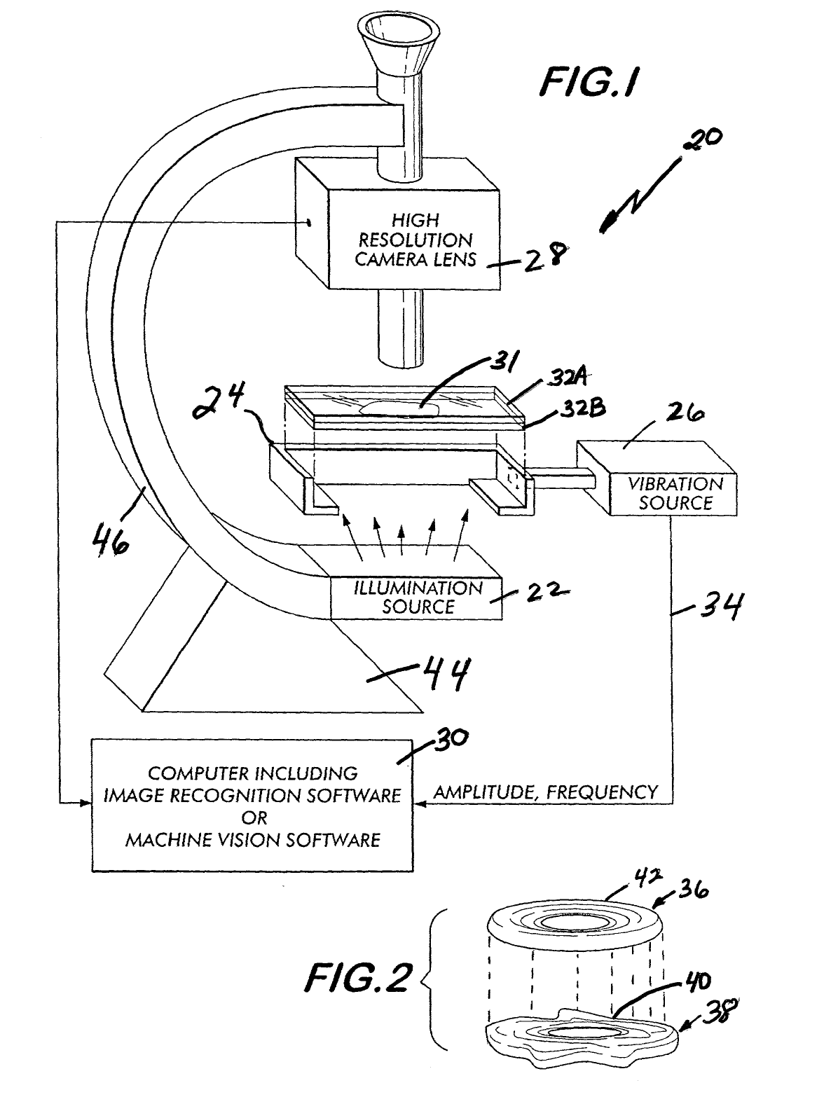

Although not limited to the structure shown in FIG. 1, the illumination source 22, high resolution camera lens 28 and the specimen support 24 may be coupled together using a single frame as in a conventional microscope, i.e., a base 44 and arm 46.

Without further elaboration, the foregoing will so fully illustrate our invention that others may, by applying current or future knowledge, readily adopt the same for use under various conditions of service.

We claim:

1. A method for determining the deformability of red blood cells (RBCs) of a living being, said method comprising the steps of:
(a) providing a sample of RBCs from the living being;
(b) applying vibratory energy to the RBCs,
(c) monitoring any distortion in the RBCs;
(d) monitoring the amplitude and frequency of said applied vibratory energy; and
(e) comparing any distortion of the RBCs against an RBC that is not subjected to said vibratory energy.

2. The method of claim 1 wherein said step of monitoring any distortion in the RBCs comprises monitoring the perimeter of an RBC subjected to vibration.

3. The method of claim 2 wherein said step of comparing any distortion of the RBC against an RBC that is not subjected to said vibratory energy comprises comparing the perimeter of the RBC that is not subjected to vibration to said perimeter of said RBC that is subjected to vibration and determining any difference between said perimeter of the RBC that is not subjected to vibration and said perimeter of said RBC that is subjected to vibration.

4. The method of claim 3 wherein said step of comparing any distortion of the RBC further comprises detecting the amplitude and frequency of vibration at which distortion of the RBC occurs.

5. An apparatus for detecting the deformability of red blood cells (RBC), said apparatus comprising:
a specimen support for supporting a sample of RBCs thereon;
a source of vibration coupled to said specimen support for applying vibratory energy to said specimen support;
an illumination source for illuminating said sample of RBCs on said specimen support;
an imaging means for obtaining an image of at least one RBC in said sample of RBCs;
a computer, coupled to said imaging means and to said vibration source, said computer having a memory comprising:
an image of an RBC that has not been subjected to vibration; and
image recognition software that compares said image of an RBC that has not been subjected to vibration with said image of at least one RBC in said sample of RBCs to determine the distortion of said at least one RBC caused by vibration.

6. The apparatus of claim 5 wherein said image recognition software determines the amplitude and frequency of vibration where distortion of said at least one RBC begins.
